(19) United States
(12) Patent Application Publication
(10) Pub. No.: US 2002/0081667 A1
(43) Pub. Date: Jun. 27, 2002

(54) Self-amplifying transcriptional amplification system

(76) Inventors: Jorn Gorlach, Durham, NC (US); Kurt Boudonck, Raleigh, NC (US)

Correspondence Address: PARADIGM GENETICS, INC, 104 ALEXANDER DRIVE, BUILDING 2, P O BOX 14528, RTP, NC, 27709-4528, US

(21) Appl. No.: 09/730,688

(22) Filed: Dec. 6, 2000

(51) Int. Cl. A01H 1/00; C12P 19/34; C12N 15/82; C12N 15/87

(52) U.S. Cl. 435/91.1; 435/320.1; 800/278

(57) ABSTRACT

A self-amplifying, transcriptional activation system is described. The system comprises

a) a first nucleic acid comprising:

iv)one or more first polynucleotide(s) comprising a DNA binding domain (DBD) and a transcription activation domain (TAD), wherein said first polynucleotide(s) encodes for and can express one or more transcription factors (TF);

v) one or more first promoter(s); and

vi) one or more first upstream activation sequence(s) (UAS1) for the first polynucleotide(s), wherein said first polynucleotide(s), said first promoter(s), and said UAS1 are operatively linked and the TF expressed by said first polynucleotide can bind to said UAS1 to repeatedly amplify its own transcription;

c) a second nucleic acid comprising

i) one or more target polynucleotide(s);

ii) one or more second promoters; and

iii) one or more second upstream activation sequence(s) (UAS2) for the target polynucleotide(s),

wherein said target polynucleotide(s), said second promoter, and said UAS2 are operatively linked, wherein the TF expressed by said first nucleic acid can also bind to UAS2 to activate the transcription of said target polynucleotide(s). The self-amplifying, transcriptional activation system is species non-specific (i.e. is not specific to a particular species) and can be used in many organism species.

### FIELD OF THE INVENTION

The present invention relates to self-amplifying, transcriptional activation system useful in a wide range of organisms (i.e. is species non-specific).

### BACKGROUND OF THE INVENTION

Recent advances in genetic engineering have provided plant and animal breeders and geneticists with the tools to insert or transform genes, which are selected portions of deoxyribonucleic acid (also known as DNA), into plants, animals and other organisms in order to produce new kinds of transgenic plants, animals and the like. Such transgenic plants and animals can have unique characteristics or traits, including resistance to pathogens such as disease organisms and insects, resistance to pesticides and enhanced stability or shelf-life of the ultimate consumer product obtained from the organism and/or improvements in the nutritional value in the edible portions thereof. Genes are made up of DNA, a complex molecule inside each cell that provides the instructions for all aspects of an organism's growth. A promoter is a region on a gene where transcription factors can bind to enable the gene to “express” itself through the production of another, but smaller molecule known as messenger RNA through a process known as transcription. Messenger RNA enables the gene to “deliver” its message or instructions to other parts of the organism's cell in many cases by being translated into a protein. Various promoters have been identified and isolated from different plant and animals. Although effective, such promoters often suffer the disadvantages of weakly expressing a desired gene or expressing a gene inconsistently or with a high degree of variability. In certain situations the promoter may disadvantageously express the gene in most of an organism's tissues, rather than being specific for a particular tissue, such as a plant or animal tissue. Further, most, if not all promoters are species specific and not effective in different species. It would be desirable to provide a transcriptional activation system that overcomes one or more of such disadvantages to provide high, reproducible levels of expression for a desired gene.

### SUMMARY OF THE INVENTION

The present invention provides a self-amplifying, transcriptional activation system, comprising:

a) a first nucleic acid comprising:

i) one or more first polynucleotide(s) comprising a DNA binding domain (DBD) and a transcription activation domain (TAD), wherein said first polynucleotide(s) encodes for and can express one or more transcription factors (TF);

ii) one or more first promoter(s); and

iii) one or more first upstream activation sequence(s) (UAS1) for the first polynucleotide(s), wherein said first polynucleotide(s), said first promoter(s), and said UAS1 are operatively linked and the TF expressed by said first polynucleotide can bind to said UAS1 to repeatedly amplify its own transcription;

b) a second nucleic acid comprising

i) one or more target polynucleotide(s);

ii) one or more second promoters; and

iii) one or more second upstream activation sequence(s) (UAS2) for the target polynucleotide(s),

wherein said target polynucleotide(s), said second promoter, and said UAS2 are operatively linked, wherein the TF expressed by said first nucleic acid can also bind to UAS2 to activate the transcription of said target polynucleotide(s).

One advantage of the present invention is that it provides a self-amplifying, transcriptional activation system which is species non-specific (i.e. is not specific to a particular species) and can be used in many organism species.

A second advantage of the present invention is that it provides a self-amplifying, transcriptional activation system which can provide transcription of a desired gene at higher or lower levels compared to known promoters.

A third advantage of the present invention is that it provides a self-amplifying, transcriptional activation system that provides transcription of a desired gene at a greater degree of reproducibility compared to known promoters.

A fourth advantage of the present invention is that it provides a self-amplifying, transcriptional activation system that can provide for transcription of a desired gene in a specific or particular tissue and/or cell type of an organism or at a particular developmental stage, or under certain environmental conditions (i.e. is inducible) at more consistent or reproducible levels compared with known promoters.

A fifth advantage of the present invention is that it provides a self-amplifying, transcriptional activation system that can reproducibly give consistent expression levels of a target gene.

### IN THE FIGURES

FIG. 1 is a generalized schematic of the self-amplifying, transcriptional activation system of the present invention.

FIG. 2 is a schematic of the self-amplifying transcriptional activation system in which the more details of a specific embodiment are depicted.

FIG. 3 is a schematic of the self-amplifying transcriptional activation system containing a promoter which is tissue specific, cell-specific, constitutive, developmental stage specific or environmental condition specific (i.e. is inducible) on a third nucleic acid or DNA construct.

FIG. 4 is a schematic of the self-amplifying transcriptional activation system which contains a promoter which is tissue specific, cell-specific, constitutive, developmental stage specific or environmental condition specific (i.e. is inducible) on the first nucleic acid or DNA construct.

FIG. 5 is a schematic of the self-amplifying transcriptional activation system which contains a tissue-specific or constitutive promoter on the second nucleic acid or DNA construct.

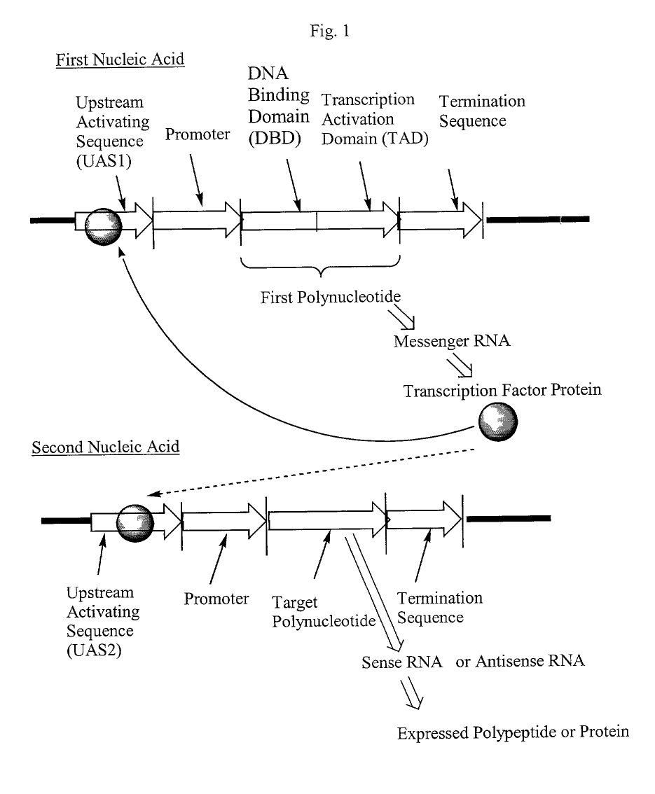

FIG. 1 shows the self-amplifying, transcriptional activation system of the present invention comprised of a first nucleic acid and a second nucleic acid. The first nucleic acid, also known as the “Loopy driver” is the “self-amplifying” portion of the overall system. The Loopy driver (i.e. the first nucleic acid) is made up of a feedforward loop to drive its own expression. The Loopy driver (the first nucleic acid) is a self-amplifying, cis-activated (i.e. autoactivated) subsystem. The Loopy driver is made up of one or more upstream activating sequences (UAS1), a promoter (such as a minimal promoter), a first polynucleotide(s), a first termination sequence, and optionally may contain other regulatory element(s) (not shown). The number of copies of upstream activating sequences (UAS) can vary. The first polynucleotide can be further characterized as having two domains: a DNA binding domain and a Transcription Activation Domain. In the Loopy driver, the first polynucleotide(s), the first promoter(s), the termination sequence and the UAS1 are operatively linked. Due to basal transcription activity or promoter/enhancer activity at the insertion site of the genome, transcription of the Loopy driver by RNA polymerase is initiated. During this reading process, the RNA polymerase transcribes or makes messenger RNA which is encoded from the first polynucleotide (including its DNA binding domain and Activation Domain). The chimeric transcription factor protein (TF) is then expressed by the first polynucleotide (i.e the messenger RNA is subsequently translated by the cell's ribosomes into a transcription factor protein). This transcription factor protein (TF) is specific in that it can bind to the UAS1 (via its DNA binding domain) and activate transcription by RNA polymerase (via its transcription activation domain). TF is expressed by the first polynucleotide, amplified (i.e. multiplied), bound again to the UAS1, and starts the cycle over again. Thus, with each cycle in the loop, each TF produced can bind to the UAS1 to repeatedly amplify its own transcription.

The second nucleic acid of the transcriptional activation system of the present invention may be viewed as the “target gene” that is “switched on” when its UAS2 portion binds with or “receives” TF that is amplified by the first nucleic acid. The second nucleic acid is made up of an upstream activation sequence (UAS2), a second promoter(s), a target polynucleotide, a second termination sequence, and optionally may contain other regulatory element(s) (not shown), wherein the UAS2, the second promoter(s), the target polynucleotide and the termination sequence are operatively linked. Moreover, it is important that the TF expressed by the first nucleic can also bind to the UAS2 of the second nucleic acid to activate transcription of the target polynucleotide. When target gene and a promoter of choice are combined with the Loopy driver, the system is able to transcribe a desired gene in a specific or particular tissue and/or cell type of an organism or at a particular developmental stage, or under certain environmental conditions (i.e. is inducible) at more consistent, reproducible levels, depending on the promoter and/or the insertion site in an organism's genome.

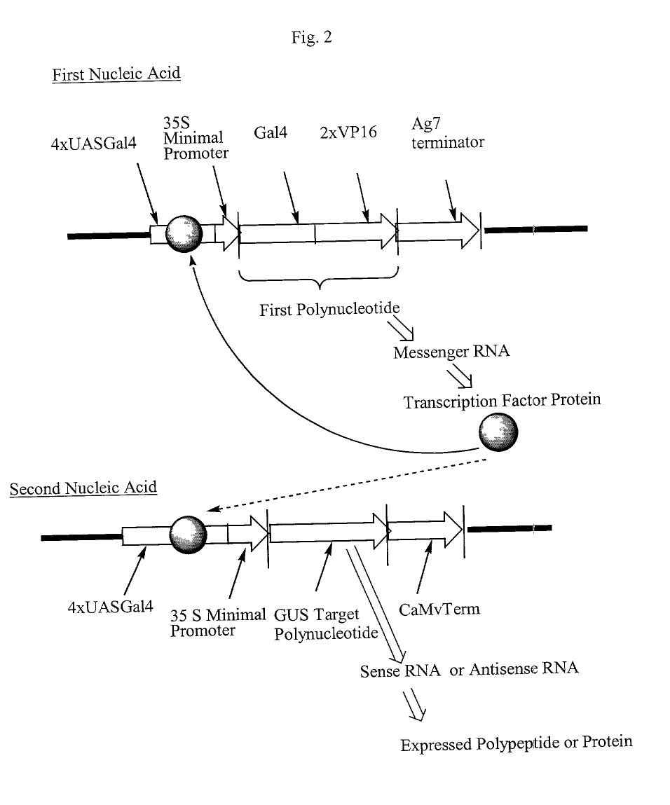

FIG. 2 depicts a specific embodiment of the self-amplifying transcriptional activation system comprised of a first nucleic acid and a second nucleic acid. In the first nucleic acid or Loopy driver, 4xUASGal4 is the Upstream Activating Sequence (UAS1), a 35S minimal promoter is the promoter, an Ag7 terminator is the termination sequence and a chimeric first polynucleotide is comprised of Gal4 DNA binding domain and a 2xVP16 transcription activation domain. In the second nucleic acid, a 4xUASGal4 is the Upstream Activating Sequence (UAS2), 35S minimal promoter is the promoter, the GUS gene is the target polynucleotide and CaMVTerm is the termination sequence. The amplified chimeric transcription factor (TF) produced by the Loopy driver can bind to the same UAS in the target construct and drive expression of the target or GUS gene. When the Loopy driver is combined with this second nucleic acid or target cassette, the Loopy driver provides consistently reproducible expression of the GUS target gene.

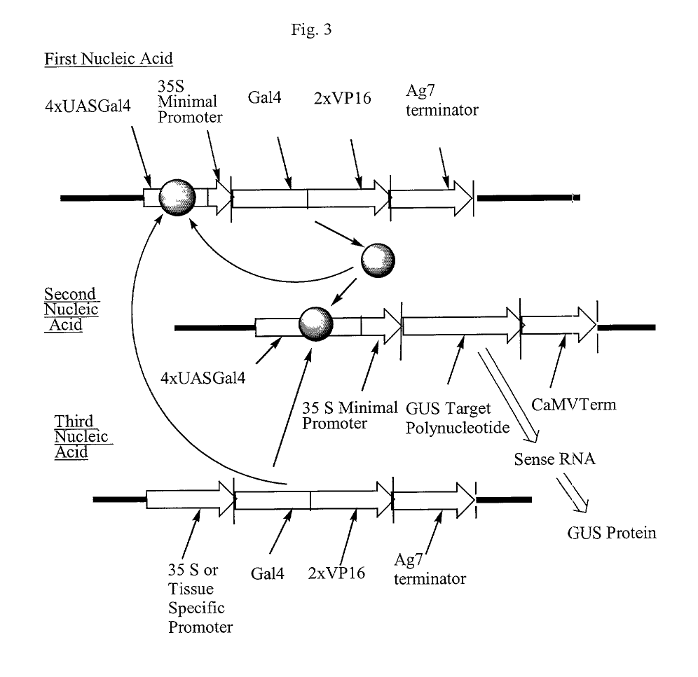

FIG. 3 depicts a specific embodiment of the self-amplifying transcriptional activation system which comprises a first nucleic acid, a second nucleic acid and a third nucleic acid or DNA construct. The third nucleic acid contains a promoter which is tissue specific, cell-specific, constitutive, developmental stage specific or environmental condition specific (i.e. is inducible). In this embodiment, the first nucleic acid (Loopy driver) and second nucleic acid (Target Gene) are described as in FIG. 2. The third nucleic acid (DNA construct) is added that contains a tissue specific or constitutive promoter such as CaMV35S, a DNA binding domain, a transcription activation domain and a termination sequence. Due to the presence of the tissue specific promoter or the constitutive promoter, the third nucleic acid can be activated to produce the chimeric transcription factor (TF) that can bind to and activate UAS1 of the Loopy Driver. At the same time, the TF can also activate the target polynucleotide. The Loopy driver amplifies and produces even more TF, activating the target gene. Thus, the Loopy Driver effectively enhances the transcriptional function of the promoter of the third nucleic acid which can be tissue specific, cell-specific, constitutive, developmental stage specific or environmental condition specific (i.e. is inducible). When the Loopy driver and Target gene are combined with the third nucleic acid that contains the tissue-specific or constitutive promoter the target gene is expressed in a tissue specific pattern (i.e. such as in a leaf or limb) at consistently reproducible levels.

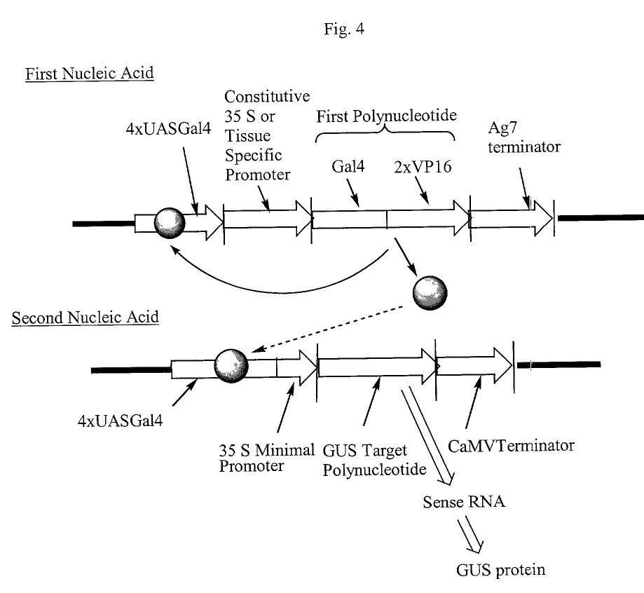

FIG. 4 depicts a specific embodiment of the self-amplifying transcriptional activation system comprised of a first nucleic acid and a second nucleic acid in which the first nucleic acid contains a promoter which is tissue specific, cell-specific, constitutive, developmental stage specific or environmental condition specific (i.e. is inducible). In the first nucleic acid (Loopy driver), 4xUASGal4 is the upstream activating sequence (UAS1), a CaMV35S promoter or a tissue specific promoter is the first promoter, the first polynucleotide is made up of a Gal4 DNA binding domain and a 2xVP16 activation domain, and an Ag7 terminating sequence. In the second nucleic acid (Target Gene) a 4xUASGal4 is the Upstream Activating Sequence (UAS2), 35S minimal promoter is the second promoter, the GUS gene is the target polynucleotide and CaMVTerm is the termination sequence. Due to the presence of the tissue specific promoter or the constitutive promoter, the first nucleic acid can activate itself to produce the chimeric transcription factor (TF) that binds to and activates UAS2 of the Target Gene. Also, the Loopy driver amplifies and produces more TF itself, further activating the target gene. The TF produced by the Loopy driver nucleic acid then binds to UAS2 and activates the target gene and expresses the target or GUS gene in a tissue specific pattern (i.e. such as in a leaf or limb) with greater tissue specificity.

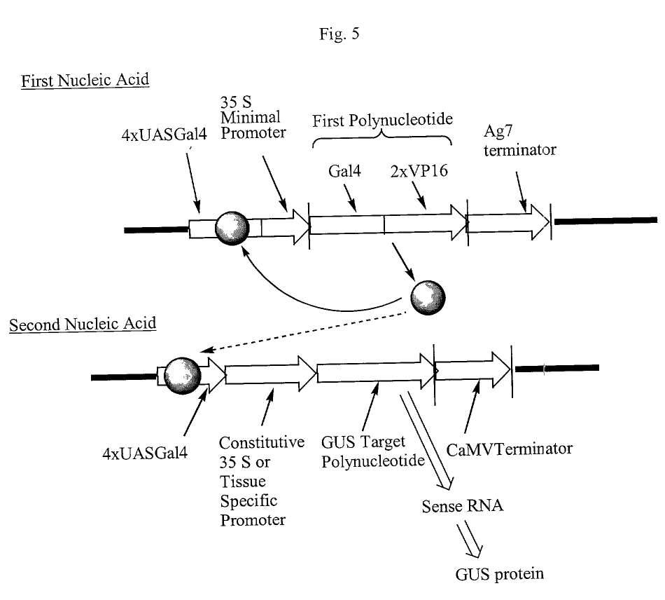

FIG. 5 depicts a specific embodiment of the self-amplifying transcriptional activation system comprised of a first nucleic acid and a second nucleic acid in which the first nucleic acid contains a minimal promoter. In the first nucleic acid (Loopy driver), 4xUASGal4 is the upstream activating sequence (UAS1), a 35S minimal promoter is the first promoter, the first polynucleotide is made up of a Gal4 DNA binding domain and a 2xVP16 activation domain, and an Ag7 terminating sequence. In the second nucleic acid (Target Gene) a 4xUASGal4 is the Upstream Activating Sequence (UAS2), a tissue specific, cell-specific, constitutive (such as 35S), developmental stage specific or environmental condition specific promoter (i.e. is inducible) is the second promoter, the GUS gene is the target polynucleotide and CaMVTerm is the termination sequence. Due to basal activity of the minimal promoter, or enhancer/promoter activity near the insertion site, the first nucleic acid can activate itself to produce the chimeric transcription factor (TF) that binds to and activates UAS2 of the Target Gene. Also, the Loopy driver amplifies and produces more TF, further activating the target gene. The TF produced by the Loopy driver nucleic acid then binds to UAS2 and activates the target gene and expresses the target or GUS gene in a tissue specific pattern (i.e. such as in a leaf or limb) with greater tissue specificity.

### DETAILED DESCRIPTION OF THE INVENTION

The present invention will now be described more fully hereinafter with reference to the accompanying figures or drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs.

“Antisense RNA” refers to a single-stranded polynucleotide that is complementary to the mRNA produced from a gene. Antisense RNA hybridizes with and inactivates mRNA.

“Chemically synthesized,” as related to a sequence of DNA, means that the component nucleotides are assembled in vitro. Chemical synthesis of DNA may be accomplished using known procedures in the art. For example, automated chemical synthesis of DNA can be performed using one of a number of commercially available apparatus or vendors.

“Coding region” is the polynucleotide or that portion of a gene that codes for a specific RNA (sense or antisense) or polypeptide (i.e. a specific amino acid sequence), and excludes the 5′ sequence which drives the initiation of transcription. The coding region is typically the first polynucleotide(s) or the target polynucleotide(s) of the first nucleic acid and second nucleic acid, respectively.

“DNA” refers to deoxyribonucleic acid.

“DNA binding domain” or “DBD” refers to the region of a polynucleotide (i.e. the first and third polynucleotides) that encodes for the polypeptide portion of the transcription factor (TF) protein that enables the TF to bind to a DNA sequence.

“Enhancer” is a DNA sequence which can stimulate promoter activity and may be an innate element of the promoter or a heterologous element inserted to enhance the level or specificity of a promoter.

“Expression” refers to the transcription of a gene or its polynucleotide region to yield sense RNA (i.e. mRNA) or antisense RNA encoded by the coding region. Expression also refers to the translation of mRNA into a polypeptide or protein.

“Gene” refers to a unit composed of a promoter region, a polynucleotide coding region and a transcription termination region, including any regulatory elements preceding or following the polynucleotide coding region.

“GUS” refers to the gene or polynucleotide coding for the enzyme, β-glucuronidase.

“Heterologous” is used to indicate that a nucleic acid sequence (e.g., a gene) or a protein has a different natural origin or source with respect to its current host. Heterologous is also used to indicate that one or more of the domains present in a protein differ in their natural origin with respect to other domains present. In cases where a portion of a heterologous gene originates from a different organism the heterologous gene is also known as a chimera.

“Homologous” is used to indicate that a nucleic acid sequence (e.g. a gene) or a protein has a similar or the same natural origin or source with respect to its current host.

“Messenger RNA,” also known as “mRNA” or “Sense RNA,” refers to a single stranded RNA molecule that specifies the amino acid sequence of one or more polypeptide chains.

“Minimal promoter” refers the minimal oligonucleotide or polynucleotide element necessary for transcription that contains a TATA-box.

“Nucleic acid” as used herein refers to DNA or RNA of genomic or synthetic origin which may be single- or double-stranded containing at least one gene that can encode for sense RNA or antisense RNA.

“Oligonucleotide” refers to a linear sequence of about 20 nucleotides or less joined by phosphodiester bonds.

“Operatively linked” generally refers to the association of various polynucleotide sequences of differing functions on a single nucleic acid or nucleic acid fragment so that the function of one polynucleotide sequence is affected by other sequence(s). In one example, with respect to the first polynucleotide(s), the first polynucleotide(s), the first promoter, the UAS1, its optional terminator sequence and any optional regulatory elements are connected in such a way that the transcription of the first polynucleotide is controlled and regulated by the UAS1 and the first promoter. In another example, with respect to the target polynucleotide(s), the target polynucleotide(s), the second promoter and the UAS2, its optional terminator sequence and any optional regulatory elements are connected in such a way that the transcription of the target polynucleotide is controlled and regulated by the UAS2 and the second promoter. In another example, a promoter is operably linked with a coding sequence (i.e. the coding sequence is under the transcriptional control of the promoter). Coding sequences can be operably linked to regulatory sequences in sense or antisense orientation.

“Polynucleotide”, also known as a “DNA sequence”, refers to a linear sequence of about 20 or more nucleotides joined by phosphodiester bonds. In the polynucleotide DNA, the sugar is deoxyribose and in RNA, ribose. The polynucleotide may be single stranded or double stranded.

“Promoter” refers to the nucleotide sequences at the 5′ end of a gene or polynucleotide which direct the initiation of transcription. Generally, promoter sequences are necessary to drive the expression of a downstream gene. The promoter binds RNA polymerase and accessory proteins, forming a complex that initiates transcription of the downstream polynucleotide sequence. The promoter can include a minimal promoter that is a short DNA sequence comprised of a TATA-box and other sequences that serve to specify the site of transcription initiation, to which regulatory elements can be added for control of expression. The promoter can also include a minimal promoter plus regulatory sequences that are capable of controlling the expression of a coding sequence or antisense RNA that is not translated. This type of promoter sequence consists of proximal and more distal upstream elements often referred to as “enhancers.” Promoters may be derived in their entirety from a native gene, or be composed of different elements derived from different promoters found in nature or even comprise synthetic DNA segments or oligonucleotides. It is understood by those skilled in the art that different promoters may direct the expression of a gene in different tissues or cell types, or at different stages of development, or in response to different environmental conditions (i.e. are inducible). Promoters which cause a gene to be expressed in most cell types at most times are referred to as constitutive promoters.

“Regulatory element” refers to polynucleotide(s) or DNA sequence(s) that play a role in determining promoter activity, i.e. a regulatory element can play a role in determining the activity of a regulatory sequence. Regulatory elements may affect the level, tissue/cell type specificity and/or developmental timing of expression. A regulatory element may be part of a promoter, or it may be located upstream or downstream of a minimal promoter. Polynucleotide sequences considered to be regulatory elements include sequences that have been shown to be target sites for binding of transcription factors, as well as sequences whose properties have not been defined but are known to have a function because their deletion from a promoter affects the expression.

“Restriction site” refers to a polynucleotide sequence at which a specific restriction endonuclease cleaves the plasmid, vector or DNA molecule.

“RNA” refers to ribonucleic acid.

“Target polynucleotide”, also known as the “second polynucleotide”, refers to a polynucleotide which encodes for sense RNA (mRNA), antisense RNA, a polypeptide or a protein of interest.

“Terminator sequence” refers to a DNA sequence downstream of, or 3′ to, a coding sequence that causes RNA polymerase to stop transcription. The terminator sequence can include a polyadenylation sequence.

“Transgenic” is an adjective describing an organism (usually a plant or animal) that contains a transgene.

“Transgene” is a gene or DNA fragment that has been stably incorporated into the genome of an organism, such as a plant or an animal.

“Transcription” is the process by which a downstream nucleotide sequence is

“read” to produce either messenger RNA (mRNA) or antisense RNA. The mRNA is the molecule that is “read” by the translational machinery to produce that protein. Variable regions at the beginning, i.e., 5′ end, and the end, i.e., 3′ end of the gene may or may not code for amino acids. Regions such as these are referred to as 5′ untranslated region (5′ UTR) and 3′ untranslated region (3′ UTR) respectively. A portion of the 5′ UTR serves as the binding region for the translational machinery (e.g., ribosomes and accessory proteins) required to synthesize a polypeptide encoded by an mRNA.

“Transcription Activation Domain” or “TAD” refers to the region of a polynucleotide (i.e. the first or third polynucleotides) that encodes for the region of the transcription factor (TF) protein that facilitates activation of transcription when the TF is contacted with a complementary Upstream Activation Sequence (UAS1).

“Transcription factor” or “chimeric transcription factor” refers to a protein required for recognition by RNA polymerases of specific stimulatory sequences in eukaryotic genes. Such proteins activate transcription by RNA polymerase when bound to upstream promoters.

“Transformation” refers to any a process by which nucleic acids are inserted into a recipient cell to effect change. Transformation may rely on known methods for the insertion of foreign nucleic acid sequences into a eukaryotic host cell. Such

“transformed” cells include stably transformed cells in which the inserted DNA is capable of replication either as an autonomously replicating plasmid or as part of the host chromosome. They also include cells which transiently express the inserted DNA or RNA for limited periods of time.

“Upstream” refers to any element to the left of, or 5′ to, the coding region for a polynucleotide.

“Upstream Activation Sequence” or “UAS ” or “UAS2 ” refers to a nucleotide sequence (activation sequence) which can bind with a corresponding TF to activate transcription of a gene. The upstream activation sequence is located “upstream” or 5′ to the coding region for a polynucleotide.

In general, transcription factors are found to contain two functional domains, one for DNA-binding and one for transcriptional activation. Transcription factors bind to and modulate the function of DNA. First, they bind specifically to their DNA-binding site, and secondly, they activate transcription. In addition, many transcription factors occur as homo- or heterodimers, held together by dimerization domains. For example, mutagenesis of the yeast transcription factors Gal4 and Gcn4 showed that their DNA-binding and transcription activation domains were in separate parts of the proteins.

The first DNA or nucleic acid (herein called LOOPY) represents, inter alia self-regulated artificial or chimeric TF composed of a DNA binding domain (DBD) and a transcriptional Activation Domain. This TF is able to activate its own transcription as well as transcription of every gene under the control of a UAS recognized by the DBD of LOOPY. In this way, LOOPY is able to amplify and activate Target gene expression. Besides others, the DBD can be derived from existing TF's like Gal4 (Schwechheimer et al., 1998), 434 repressor (Xu et al., 2000), Vp1 (McCarty et al., 1991), lac repressor (Moore et al., 1998), LexA (Schwechheimer et al., 1998), bZIP factors (Sprenger-Haussels et al., 2000) or artificial DNA binding domains like mutated DBD from general DNA binding proteins and other TFs. The transcriptional Activator Domain can be derived from existing Transcriptional Activators like Gal4 (Schwechheimer et al., 1998), VP16 (Schwechheimer et al., 1998), Gln3p (Svetlov et al., 1997), THM18 (Schwechheimer et al., 1998), TBP (Xiao et al., 1995) or artificial peptides able to activate transcription.

Depending on the line, organism, choice of UAS, promoter, place of insertion in the genome, regulatory elements, DNA binding factor, activation factor and other variables, Loopy driver can amplify itself to a steady state level. For example, the system containing the Loopy driver can be expected to reach its plateau levels and stay there. Thus, when combined with other DNA constructs or “second nucleic acids” the Loopy driver can advantageously drive expression of target genes of interest.

In the self-amplifying, transcriptional activation system of the present invention, the first nucleic acid (Loopy driver) and the second nucleic acid (target gene) can be inserted into separate plasmids, i.e. a “driver plamid” and a “target plasmid,” prior to insertion into the organism. However, the system can also be designed or constructed so that the first nucleic acid and the second nucleic acid and/or cassettes contains these nucleic acids, are on the same plasmid (i.e. one plasmid).

Thus, the self-amplifying, transcriptional activation system of the present invention can provide tissue specific, cell-specific, constitutive, developmental stage specific or environmental condition specific (i.e. inducible) expression. Such specificity can be obtained through insertion of the Loopy driver cassette close to a promoter/enhancer in the genome. Alternatively, such specificity can be obtained through combination with a promoter that is tissue specific, cell-specific, constitutive, developmental stage specific or environmental condition specific (i.e. inducible) on a single cassette or plasmid or on separate cassettes or plasmids.

The self-amplifying, transcriptional activation system of the present invention has the further advantage of being species non-specific. Thus, the system can be utilized in a wide range of organisms. Such organisms can be from any of the six kingdoms:

Monera-Prokaryotic cells. Includes organisms such as bacteria, blue-green algae. Bacteria genera include, but are not limited to Bacillus and Streptomyces.

Protista-Eukaryotic cells. Includes organisms such as amoebae, euglena, paramecium and diatoms

Fungi-Eukaryotic cells. Includes organisms such as mushrooms, water molds, bread molds (filamentous fungi). Fungi genera include, but are not limited to Magnaporthe, Mycosphaerella, Botrytis, Saccharomyces, Aspergillus, Puccinia, Erysiphe, Ustilago, Fursarium, Phytophthora and Penicillium. Specific examples of fungi include species (sp.) such as Magnaporthe grisea, Mycosphaerella graminicola, Botrytis cinerea, Saccharomyces cerevisiae (yeast), Aspergillus fumigatus and Aspergillus niger.

Plantae-Eukaryotic cells. Includes organisms such as herbs, shrubs, trees, monocotyledonous and dicotyledenous plants. Monocotyledonous plants include rice, wheat, corn, barley and oats. Dicotyledenous plants, include but are not limited to Arabidopsis, soybean, peanut, alfalfa, tomato, eggplants, potatoes, cabbage, turnips, rapeseed, apples, pears, berries, cucumbers, beets and carrots.

Animalia-Eukaryotic cells. Includes organisms such as invertebrates and vertebrates. Invertebrates include nematodes, mollusks and insects such as Drosophila. Vertebrates include, birds and mammals such as mice and humans.

Archae-Includes cells such as archae.

Preferably, the organism is a plant. Thus, the present invention is directed toward transgenic plants containing the self-amplifying, transcriptional amplification system described herein, together with seeds or plant parts obtained from the transgenic plant. Progeny of the transgenic plant may also be obtained by either a sexual or asexual cycle.

Thus, the present invention is directed toward a method of expressing a target polynucleotide in a plant cell, by introducing into a plant the transcriptional activation amplification system described herein. There are various methods for producing such transgenic plants. In one method, the transgenic plant is prepared by:

a) providing first and second plants, wherein the first plant comprises said first nucleic acid and the second plant comprises said second nucleic acid described herein;

b) either pollinating the first plant with pollen from the second plant or pollinating the second plant with pollen from the first plant to produce a transgenic embryo or seed containing the self-amplifying, transcriptional amplification system described herein; and

c) growing the embryo or seed into a plant.

In another method a transgenic plant can be prepared by:

a) transforming a first plant with a construct containing the first nucleic acid to give a transformed plant whose seed contains the first nucleic acid;

b) growing said seed containing the first nucleic acid into a plant; and

c) transforming the plant containing the first nucleic acid with a construct containing the second nucleic acid to give a transformed plant whose seed contains the self-amplifying, transcriptional amplification system described herein.

In order to improve the ability to identify transformants, one may desire to employ a selectable or screenable marker gene as, or in addition to, the target polynucleotide or on the plasmid containing the Loopy driver. “Marker genes” are genes that impart a distinct phenotype to cells expressing the marker gene and thus allow such transformed cells to be distinguished from cells that do not have the marker. Such genes may encode either a selectable or screenable marker, depending on whether the marker confers a trait which one can ‘select’ for by chemical means, i.e., through the use of a selective agent (e.g., a herbicide, antibiotic, or the like), or whether it is simply a trait that one can identify through observation or testing, i.e., by ‘screening’ (e.g., the GUS gene). Of course, many examples of suitable marker genes are known to the art and can be employed in the practice of the invention. The selectable marker gene may be the only heterologous gene expressed by a transformed cell, or may be expressed in addition to another heterologous gene transformed into and expressed in the transformed cell. Selectable marker genes are utilized for the identification and selection of transformed cells or tissues. Selectable marker genes include genes encoding antibiotic resistance, such as those encoding neomycin phosphotransferase II (NEO) and hygromycin phosphotransferase (HPT), as well as genes conferring resistance to herbicidal compounds. Herbicide resistance genes generally code for a modified target protein insensitive to the herbicide or for an enzyme that degrades or detoxifies the herbicide in the plant before it can act. See, DeBlock et al., EMBO J. 6, 2513 (1987); DeBlock et al., Plant Physiol. 91, 691 (1989); Fromm et al., BioTechnology 8, 833 (1990); Gordon-Kamm et al., Plant Cell 2, 603 (1990). For example, resistance to glyphosphate or sulfonylurea herbicides has been obtained using genes coding for the mutant target enzymes, 5-enolpyruvylshikimate-3-phosphate synthase (EPSPS) and acetolactate synthase (ALS). Resistance to glufosinate ammonium, boromoxynil, and 2,4-dichlorophenoxyacetate (2,4-D) have been obtained by using bacterial genes encoding phosphinothricin acetyltransferase, a nitrilase, or a 2,4-dichlorophenoxyacetate monooxygenase, which detoxify the respective herbicides.

In a preferred embodiment of the invention, recipient cells for transformation are plant cells, preferably monocot plant cells, more preferably dicot plant cells, even more preferably Arabidopsis species plant cells, and most preferably Arabidopsis thaliana plant cells. “Plant cells” as used herein includes plant cells in plant tissue or plant tissue and plant cells and protoplasts in culture. Plant tissue includes differentiated and undifferentiated tissues of plants, including but not limited to, roots, shoots, leaves, pollen, seeds, tumor tissue and various forms of cells in culture, such as single cells, protoplasts, embryos and callus tissue. The plant tissue may be in plant, or in organ, tissue or cell culture. Plant parts include attached or detached portions of a plant, including leaves, stems, roots, flowers, fruits or parts thereof.

The following Examples are provided to illustrate the present invention, and should not be construed as limiting thereof.

### EXAMPLE 1

### Preparation and Testing of the Self-Amplifying Transcriptional System

A dicotyledenous plant, known as Arabidopsis, is used to test the self-amplifying, transcriptional activation system. The second nucleic acid (Target gene), is inserted into an Arabidopsis plant by transforming an Arabidopsis plant line with a bacteria known as Agrobacterium containing a DNA construct including the second nucleic acid. The treated plants are self-fertilized to give a homozygous target line containing the second nucleic acid (GUS gene). This target line containing the second nucleic acid or GUS gene serves as the control plant that do not contain the Loopy driver. The second nucleic acid (Target gene) is described in FIG. 2 wherein the promoter is a Nos101 minimal promoter and the target polynucleotide encodes for the GUS protein. The first nucleic acid (Loopy driver) is described in FIG. 2, where the promoter used is a CaMV35S-60 minimal promoter SEQ ID NO:1. The first nucleic acid also includes a herbicide selection marker (not shown) which is a gene used to make the plant resistant to a specific herbicide. The first nucleic acid (driver gene) is inserted into the target line by contacting the reproductive parts of the Arabidopsis plant with a bacteria known as Agrobacterium which contains the first nucleic acid. The bacteria insert the first nucleic acid into female reproductive tissue, giving a T1 or transformed seed. However, only a small portion of the T1 seed will be transgenic for the Loopy driver, i.e. the seed contains both the first nucleic acid and the second nucleic acid. To select those seeds that are transgenic, the seeds are grown in media containing the selectable herbicide. Seedlings which survive in the presence of herbicide are t-ansgenic. Seedlings are stained with a solution known as X-gluc and the plants are examined for the presence of blue color, an indication of the activity of the GUS gene. The strong blue color observed in the plant seedlings indicates the self-amplifying, transcriptional activation system using Loopy driver is operating to express the GUS gene.

### EXAMPLE 2

### Preparation and Testing of the Self-Amplifying Transcriptional System

Essentially the same procedure as in Example 1 is followed except that the first nucleic acid (Loopy driver) uses a different artificial minimal promoter SEQ ID NO:2. A more diffuse blue pattern is obtained, also indicating the self-amplifying, transcriptional activation system using Loopy driver is operating to express the GUS gene. Also, by using a different minimal promoter, the self-amplifying, transcriptional activation system can be regulated differently.

### Preparation of Starting Materials

Starting materials or components or elements that can be used to prepare the self-amplifying, transcriptional activation system of the present invention are known in the art, as taught or exemplified in the following references and to references cited therein, whose preparative teachings are incorporated herein by reference:

Moore I, Galweiler L, Grosskopf D, Schell J, Palme K (1998). A transcription activation system for regulated gene expression in transgenic plants. Proc Natl Acad Sci U S A, January 6;95(1):376-81; McCarty D R, Hattori T, Carson C B, Vasil V, Lazar M, Vasil I K (1991). The Viviparous-1 developmental gene of maize encodes a novel transcriptional activator. Cell, September 6;66(5):895-905;

Schwechheimer, C., Smith, C. and Bevan, M. W. (1998). The activities of acidic and glutamine-rich transcriptional activation domains in plant cells: design of modular transcription factors for high-level expression. Plant Mol. Biol. 36, 195-204;

Sprenger-Haussels M, Weisshaar B (2000). Transactivation properties of parsley proline-rich bZIP transcription factors. Plant J, April;22(1):1-8;

Svetlov V, Cooper T G (1997). The minimal transactivation region of Saccharomyces cerevisiae Gln3p is localized to 13 amino acids. J Bacteriol, December;179(24):7644-52;

Xiao H, Friesen JD and Lis JT (1995). Recruiting TATA-binding protein to a promoter: transcriptional activation without an upstream activator. Mol. Cellul. Biol., October: 5757-5761; and

Xu J, Koudelka G B (2000). Mutually exclusive utilization of P(R) and P(RM) promoters in bacteriophage 434 O(R). J Bacteriol, June;182(11):3165-74.

What is claimed is:

1. A self-amplifying, transcriptional activation system, comprising:
a) a first nucleic acid comprising:
i) one or more first polynucleotide(s) comprising a DNA binding domain (DBD) and a transcription activation domain (TAD), wherein said first polynucleoticle(s) encodes for and can express one or more transcription factors (TF);
ii) one or more first promoter(s); and
iii) one or more first upstream activation sequence(s) (UAS1) for the first polynucleotide(s), wherein said first polynucleotide(s), said first promoter(s), and said UAS1 are operatively linked and the TF expressed by said first polynucleotide can bind to said UAS1 to repeatedly amplify its own transcription;
b) a second nucleic acid comprising
i) one or more target polynucleotide(s);
ii) one or more second promoters; and
iii) one or more second upstream activation sequence(s) (UAS2) for the target polynucleotide(s),
wherein said target polynucleotide(s), said second promoter, and said UAS2 are operatively linked, wherein the TF expressed by said first nucleic acid can also bind to UAS2 to activate the transcription of said target polynucleotide(s).

2. The self-amplifying, transcriptional amplification system of claim 1, wherein the first nucleic acid comprises a first promoter that is a minimal promoter, a constitutive promoter, a tissue-specific promoter, a cell type specific promoter, a developmental stage promoter, an environmental condition specific promoter or an inducible promoter.

3. The self-amplifying, transcriptional amplification system of claim 1, wherein the second nucleic acid comprises a second promoter that is a minimal promoter, a constitutive promoter, a tissue-specific promoter, a cell type specific promoter, a developmental stage promoter, an environmental condition specific promoter or an inducible promoter.

4. The self-amplifying, transcriptional amplification system of claim 1, wherein the DBD is selected from the group consisting of Gal4, 434-repressor, lac-repressor, Vp1, bZIP factors, LexA, and synthetic DBD's.

5. The self-amplifying, transcriptional amplification system of claim 1, wherein the TAD is selected from the group consisting of Gal4, VP16, Gln3p, THM18, TBP, and synthetic peptides.

6. The self-amplifying, transcriptional amplification system of claim 1, wherein DBD is selected from the group consisting of Gal4, 434-repressor, lac-repressor, Vp1, bZIP factors, Lex A and artificial DNA binding domains; and the TAD is selected from the group consisting of Gal4, VP16, Gln3p, THM18, TBP, and synthetic peptides.

7. The self-amplifying, transcriptional amplification system of claim 1 wherein the first nucleic acid comprises a DBD which is Gal4, a TAD which is 2xVP16, a promoter which is a minimal promoter and a UAS1 which is 4xUASGal4; and the second nucleic acid comprises a target polynucleotide, a second promoter which is a minimal promoter and a UAS2 which is 4xUASGal4.

8. The self-amplifying, transcriptional amplification system of claim 1, wherein said first nucleic acid and said second nucleic acid further comprise a termination sequence operatively linked to said first polynucleotide and said target polynucleotide, respectively.

9. The self-amplifying, transcriptional amplification system of claim 1, wherein the first nucleic acid, the second nucleic acid or both the first nucleic acid and the second nucleic acid further comprise one or more regulatory element(s).

10. The self-amplifying, transcriptional amplification system of claim 1 further comprising c) a third nucleic acid, the third nucleic acid comprising:
i) one or more promoters; and
ii) one or more first polynucleotide(s) comprising a DNA binding domain (DBD) and a transcription activation domain (TAD), wherein said third polynucleotide(s) encodes for and can express one or more transcription factors (TF), wherein the TF expressed by said third nucleic acid can also bind to UAS1 and UAS2 to activate self-amplification by said first polynucleotide(s).

11. The self-amplifying, transcriptional amplification system of claim 1, wherein the TF comprises SEQ ID NO: 3.

12. The self-amplifying, transcriptional amplification system of claim 1, wherein the UAS1 comprises SEQ ID NO:4.

13. A transgenic plant containing the self-amplifying, transcriptional amplification system of claim 1.

14. Seeds or plant parts obtained from the transgenic plant of claim 13.

15. Progeny of the transgenic plant of claim 13, produced by sexual or asexual cycle.

16. A method of producing the transgenic plant of claim 13 comprising:
a) providing first and second plants, wherein the first plant comprises said first nucleic acid and the second plant comprises said second nucleic acid of claim 1;
b) either pollinating the first plant with pollen from the second plant or pollinating the second plant with pollen from the first plant to produce a transgenic embryo or seed containing the self-amplifying, transcriptional amplification system of claim 1; and
c) growing the embryo or seed into a plant.

17. A method of producing the transgenic plant of claim 13 comprising:
a) transforming a first plant with a construct containing said first nucleic acid to give a transformed plant whose seed contains said first nucleic acid;
b) growing said seed containing said first nucleic acid into a plant;
c) transforming said plant containing said first nucleic acid with a construct containing said second nucleic acid to give a transformed plant whose seed contains the self-amplifying, transcriptional amplification system of claim 1.

18. A method of expressing a target polynucleotide in a plant cells comprising introducing into a plant the transcriptional activation amplification system of claim 1.


SEQUENCE LISTING (4 sequences)

SEQ ID NO: 1 - DNA, 82 bp, cauliflower mosaic virus
cccccactatccttcgcaagacccttcctctatataaggaagttcatttcatttggagagaacacgggggatcgggtatcga

SEQ ID NO: 2 - DNA, 57 bp, Artificial Sequence
  Artifical minimal promoter
ctgcagtcctctatataaggaggggttcattcccatttgaaggatcaatagtttaaa

SEQ ID NO: 3 - DNA, 941 bp, Artificial Sequence
  chimeric transcription factor from yeast, Gal4 and Herpes simplex VP16
aagcttcatatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcggatccggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgactccgagtttgagcagatgtttaccgatgcccttggaattgacgagtacggtgggctagatccggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgactccgagtttgagcagatgtttaccgatgcccttggaattgacgagtacggtgggctgaattc

SEQ ID NO: 4 - DNA, 88 bp, Artificial Sequence
  Synthetic sequence for Upstream Activation Sequence or UAS1
gatccggaagactctcctccgagatccggaagactctcctccgagatccggaagactctcctccgagatccggaagactctcctccga